(19) United States
(12) Patent Application Publication
(10) Pub. No.: US 2004/0172671 A1
(43) Pub. Date: Sep. 2, 2004

(54) Transgenic plants protected against parasitic plants

(76) Inventors: Radi Ali, Upper Galilee (IL); James H. Westwood, Blacksburg, VA (US); Carole Cramer, Blacksburg, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/466,436

(22) PCT Filed: Jan. 25, 2002
(86) PCT No.: PCT/US02/22520

Related U.S. Application Data
(60) Provisional application No. 60/264,073, filed on Jan. 26, 2001.

(51) Int. Cl. A01H 1/00; C12N 15/82

(52) U.S. Cl. 800/278; 800/279

(57) ABSTRACT

Transgenic plants which are resistant to damage from parasitic plants and methods for generating such transgenic plants are provided. The transgenic plants are genetically engineered to contain a lytic toxin gene such as the cecroipin gene sarcotoxin IA. Expression of the gene is driven by an inducible promoter such as a parasite induced promoter, or a promoter which selectively expresses the gene in a particular region of the plant, e.g. the root system.

### BACKGROUND OF THE INVENTION

1. Field of the Invention

The invention generally relates to the production of plant varieties that are resistant to parasitic plants. In particular, the invention provides methods for producing host plants which express a cecropin protein such as sarcotoxin IA, or other lytic toxins, rendering the host plants resistant to parasitic plants.

2. Background of the Invention

Parasitic plants are destructive agricultural pests. With respect to their biology, parasitic plants form a physiological continuum to a host plant such that it is able to augment its own nutrition at the expense of the other plant. Thus, it is not surprising that many of the more than 3,000 species of parasitic angiosperms are economically important weeds. Indeed, certain parasites are among the most destructive of weeds known ENRfu(Parker and Riches 1993; Sauerborn 1991).

Parasitic plants vary widely in their degree of dependence on the host. Some are photosynthetic and have the ability to survive without a host, but are able to take advantage, of an available host to augment their nutrition (facultative parasites, i.e. Triphysaria spp.). Others have an absolute requirement for a host, but retain some photosynthetic capacity (obligate hemiparasites, i.e. Stiriga and Alectra spp., mistletoes and some Cuscuta spp.). In the final category are those parasites that lack any photosynthetic capacity [indeed, some have lost much of their chloroplast genomes ENRfu(DePamphilis and Palmer 1990; DePamphilis et al. 1997)], and are completely reliant on the host for all nutritional needs. This last category (obligate holoparasites) represents the most extreme example of parasitism, and it is to this group that Orobanche and some Cuscuta spp. belong.

The parasitic weed Orobanche spp. (broomrape) is an obligate holoparasite that attacks the roots of many economically important crops throughout the semiarid regions of the world, especially the Mediterranean and Middle East, where Orobanche is endemic. The genus Orobanche has more than 100 species, with five (O. aegyptiaca, O. ramosa, O. minor, O. cernua, and O. crenata), being considered significant parasites of crops. In Israel and in the Middle East, Orobanche spp. attack members of the Solanaceae, Fabaceae, Compositae, Umbelliferae, and more than 30 other food and ornamental crops causing severe losses in yield and quality (Parker and Wilson, 1986; Parker and Riches, 1993). Annual food crop losses from this weed in the Middle East can be conservatively estimated at $1.3 billion to $2.6 billion.

Although many of the most destructive parasitic weeds (Striga and Orobanche) primarily impact other regions of the world, parasitic weeds are clearly a concern to US agriculture. Surveys of university herbaria have indicated numerous past introductions of O. minor into the US ENRfu(Frost and Musselman 1980) and led to the discovery of existing infestations in Virginia, North Carolina, South Carolina, Georgia (English et al. 1998) and Oregon. At one point Striga asiatica established an infestation area covering much of the Carolinas and required many years and more than $200 million to bring under control (Eplee and Langston, 1991). Personnel from USDA/APHIS continue to expend time and resources to eradicate infestations of O. minor and O. ramosa, as well as the remaining Striga, and constant vigil is required to prevent the establishment of new infestations.

Cuscuta spp. (principally C. campestris, but including several other species) is a stem parasite and an important weed in Europe, the Middle East, Africa, North America and South America (Parker and Riches, 1993). It attacks and damages a wide variety of crops including forage crops such as lucerne, and red clover, vegetables such as asparagus, carrot, chickpea, grapevine, honeydew melon, lespedeza, onion, potato, red beet tomato and eggplant. Sugarbeet and faba bean are also parasitized, as are some tree crops (coffee) and ornamentals. Estimates of forage crops loss range from 20 to 57%, and sugarbeet yields reduced by 3.5-4 ton/ha.

The dwarf mistletoes (Arceuthobium spp.) are parasites of coniferous trees in the United States, Canada, Mexico, Central America and Asia. Hosts include Pinus, Picea spp., Douglas fir, and Western hemlock. It's estimated that over 50% of forests in the Western US are infested, with losses of volume growth estimated up to 65% in severe infestations. Leafy mistletoes (Phoradendron and Viscum spp.) are distributed world-wide and attack both fruit and forest trees. These may weaken trees and leave them susceptible to other pathogens, but are less destructive than the dwarf mistletoes.

Parasitic weeds such as Orobanche and Striga are difficult to control because they are closely associated to the host root and are concealed underground for most of their life cycle. The parasites are not controlled effectively by traditional cultural or herbicidal weed control strategies (Foy et al. 1989). Currently in Israel and throughout the Middle East, the best control method is to kill seeds in the soil by fumigation with methyl bromide (Jacobson 1994). This method is expensive, laborious, and extremely hazardous to the environment (methyl bromide use is being phased out by international agreement to protect the global environment). The development of herbicide-resistant crops has recently offered another Orobanche control approach, based on herbicide translocation through the host plant to the parasite (Surov et al. 1998; Joel et al. 1995). However, this approach depends on commercial availability of herbicide-resistant crops, requires correct application of chemicals, and may be countered by the development of herbicide-resistant populations of the parasite (Gressel et al. 1996). The best long-term strategy for limiting damage by Orobanche is the development of Orobanche-resistant crops (Cubero, 1991; Ejeta et al., 1991).

Control methods for Cuscuta include hand-pulling (involves loss/damage of host tissue), crop rotation to non-hosts (but other weeds must also be controlled), close mowing of forages, burning, and herbicides. Little work has been done on identifying resistant varieties of susceptible crops.

Methods for control of mistletoes include pruning (not practical in forestry situations) and forest management (selective thinning, burning). Herbicides are of little use, and few species show significant varietal resistance that could be used in a breeding program.

As mentioned above, the best long-term strategy for controlling parasitic weeds may be through the identification and breeding of resistant genotypes. Parasite-resistant crops offer several advantages over other control measures, such as reduced labor, less expense, increased cropping choices, and elimination of the need for chemicals that may be harmful to the environment. However, despite many years of hard work by plant breeders, resistant cultivars of most crops are not available.

It would be highly desirable to have available varieties of plants, especially crop plants for food production, which are resistant to parasitic plants. The availability of such plant varieties would lessen or eliminate the need for alternative parasitic plant eradication measures, while increasing crop yields.

### SUMMARY OF THE INVENTION

It is an object of this invention to provide a transgenic plant protected from parasitic plants. The transgenic plant is comprised of a host plant harboring an expressible gene encoding a lytic toxin that inhibits attack from parasitic plants. The transgenic plant may be a dicotyledon such as a tomato, a potato, or tobacco. The lytic toxin gene which is expressed may be a member of the cecropin family, and exemplary members of which is sarcotoxin IA, as represented by SEQ ID NO:1.

Parasitic plants to which resistance may be developed include Orobanche spp., Striga spp., Alectra spp., Cuscuta spp., Arceutiobium spp., Phoradendron spp., and Viscum spp. In a preferred embodiment, the parasitic plant is of the genus Orobanche (e.g. Orobanche aegyptiaca and Orobanche minor).

The transgenic plant of the present invention may further comprise an inducible promoter that is operatively linked to the expressible lytic toxin gene. The promoter regulates localized expression of the lytic toxin in the area of invasion of said parasitic plant, and may be, for example, a parasite inducible promoter. Further, the promoter may be selectively active in one area of the plant such as the root system. In a preferred embodiment of the invention, the inducible promoter is located upstream of the expressible lytic toxin gene, and preferably within one hundred base pairs of the expressible gene.

The present invention also provides a method for protecting plants from damage caused by parasitic plants The method comprises providing a host plant with an expressible gene encoding a lytic toxin which produces a polypeptide that inhibits attack from parasitic plants. The method may further include providing an inducible promoter which regulates localized expression of the lytic toxin gene in the area of invasion of said parasitic plant.

The present invention also provides a method of preventing or reducing damage in a host plant which may be attacked by a parasitic plant. The method comprises the step of harboring in the host plant an expressible gene encoding a lactic toxin that inhibits attack from parasitic plants. The host plant may be a dicotyledon such as a tomato, a potato, or tobacco. The lytic toxin gene which is expressed may be a member of the cecropin family, an exemplary member of which is sarcotoxin IA, as represented by SEQ ID NO:1.

Parasitic plants to which resistance may be developed include Orobanche spp., Striga spp., Alectra spp., Cuscuta spp., Arceuthobium spp., Phoradendron spp., and Viscum spp. In a preferred embodiment, the parasitic plant is of the genus Orobanche (e.g. Orobanche aegyptiaca andOrobanche minor).

The method may further comprise providing an inducible promoter that is operatively linked to the expressible lytic toxin gene. The promoter regulates localized expression of the lytic toxin in the area of invasion of said parasitic plant, and may be for example a parasite induced promoter. Further, the promoter may be selectively active in one area of the plant such as the root system. In a preferred embodiment of the invention, the inducible promoter is located upstream of the expressible lytic toxin gene, and preferably within one hundred base pairs of the expressible gene.

The present invention further provides potato, tomato and tobacco plants transformed by an expressible gene encoding a lytic toxin which produces a polypeptide that inhibits attack from parasitic plants. In a preferred embodiment, the expressible gene is the sarcotoxin IA gene as represented by SEQ ID NO: 1.

### BRIEF DESCRIPTION OF THE DRAWINGS

FIG. 1A and B. Sarcotoxin IA sequence. A. Nucleic acid sequence of Sarcotoxin IA gene (SEQ ID NO:1). B. Amino acid sequence of Sarcotoxin IA polypeptide (SEQ ID NO:2).

FIG. 2. Schematic depiction of construct containing the root-specific promoter (Tob), the translation enhancing sequence Ω, sarcotoxin coding sequences and the nopaline synthase (Nos) terminator. The construct was cloned into a pGA492 binary vector and transformed to Agrobacterium for plant transformation.

FIG. 3. Tobacco NN plants which were transformed and non-transformed with sarcotoxin gene were analyzed for broomrape early emergence, total parasites and tobacco yield. All data were analyzed by analysis of variance and means were separated using Duncan's new multiple range test at the 0.05 significance level.

### DETAILED DESCRIPTION OF THE PREFERRED EMBODIMENTS OF THE INVENTION

Applicants have discovered that, surprisingly, expression of a lytic toxin in transgenic host plants renders the host plants resistant to parasitic plants. The lytic toxin is selectively toxic to parasitic plants when synthesized in host tissue invaded by the parasite, i.e. expression of the gene is not detrimental to the host plant. The development of transgenic plant varieties expressing lytic toxins obviates the need for other less desirable and less effective types of parasitic plant eradication procedures and promotes crop productivity in a cost effective manner.

In a preferred embodiment of the instant invention, the lytic toxin is a cecropin. Cecropins comprise a family of small basic polypeptides that have been isolated from the hemolymph of insects (Boman et al. 1987). These proteins possess antibacterial activity and are important in the immune response of various insects. Sarcotoxin IA, a 40-residue peptide, is one of four cecropin-type proteins encoded by the sarcotoxin I gene cluster in the flesh fly, Sarcophaga peregrina (Kanai et al. 1989). The primary target of this toxin is assumed to be the microbial membrane, and its antimicrobial effect is probably due to ionophore activity (Natori, 1995; Okada et al. 1985). A cDNA clone of sarcotoxin IA was isolated and characterized by Kanai and Natori (1989). Toxicity studies on a variety of cell types have shown that, although plant protoplasts are more sensitive to cecropins than are animal cells, plant cells are one to two orders of magnitude less sensitive to the toxin than their bacterial pathogens (Jaynes et al. 1989; Nordeen et al. 1992). It has subsequently been shown that sarcotoxin genes can be used to engineer plants for resistance to bacterial pathogens (During, 1996).

Until recently, potent cecropin peptides were either isolated from the hemolymph of flies or were synthesized in vitro; production and isolation of active cecropins by heterologous microorganisms has not been reported. Recently, the sarcotoxin IA gene has been expressed in Saccharomyces cerevisiae under the control of a constitutive phosphoglycerate kinase (PGK) yeast promoter (Minet et al. 1992). The sarcotoxin-like peptide (SLP) was secreted from yeast cells and had a potent cytotoxic effect against several bacteria, including plant pathogenic bacteria, similar to the toxic effects of the authentic sarcotoxin IA (Aly et al, 1999).

We have now shown that a gene encoding the cecropin sarcotoxin IA polypeptide can be inserted into and functionally expressed in transgenic host plants, expression of the polypeptide surprisingly confers on the host plant resistance to parasitic plants. Further, in order to achieve appropriate levels of expression, the gene is fused to a promoter which regulates localized expression of the gene in the area of invasion of said parasitic plant. Thus, localized, intense expression of the polypeptide occurs at the site of invasion.

As used herein, the term “lytic peptide” includes any polypeptide which lyses the membrane of a cell in an in vivo or in vitro system in which such activity can be measured. Exemplary lytic peptides include lysozymes, cecropins, attacins, melittins, magainins, bombinins, xenopsins, caeruleins, the polypeptide from gene 13 of phage P22, S protein from lambda phage, E protein from phage PhiX174, and the like. Preferred lytic peptides have from about 30 to about 40 amino acids, at least a portion of which are arranged in an amiphiphilic alpha-helical conformation having a substantially hydrophilic head with a positive charge density, a substantially hydrophobic tail, and a pair of opposed faces along the length of the helical conformation, one such face being predominantly hydrophilic and the other being predominantly hydrophobic. The head of this conformation may be taken as either the amine terminus end or the carboxy terminus end, but is preferably the amine terminus end.

Suitable lytic peptides generally include cecropins such as cecropin A, cecropin B, cecropin D, lepidopteran, deftericin, coleoptericin, apidaecin and abaecin; sarcotoxins such as sarcotoxin IA, sarcotoxin IB, and sarcotoxin IC; and other polypeptides such as attacin and lysozyme obtainable from the hemolymph of any insect species which have lytic activity against bacteria and fungi similar to that of the cecropins and sarcotoxins. It is also contemplated that lytic peptides may be obtained as the lytically active portion of larger peptides such as certain phage proteins such as S protein of lambda phage, E protein of Phix174 phage and P13 protein of P22 phage; and C9 protein of human complement. As used herein, classes of lytically active peptides such as, for example, “cecropins,” “attacins” and “phage proteins,” and specific peptides within such classes, are meant to include the lytically active analogues, homologues, fragments, precursors, mutants or isomers thereof unless otherwise indicated by context. Any lytic toxin that can be used to create a transgenic plant that is resistant to parasitic plants due to expression of the corresponding protein may be utilized in the practice of the present invention.

Several antibiotic peptides have been also isolated from amphibia, e.g., magainin (Zasloff, 1987), ranalexin (Clark., D. P. et al., 1994), brevinins (Morikawa, N. et al., 1992) and esculantins (Simmaco, M. et al., 1993). The mechanism of action of these is similar to that of cecropin, i.e., the formation of ion channels in the lipid membrane of bacteria to rupture the cell.

Further discussion of lytic peptides can be found, for example, in U.S. Pat. No. 5,597,945 which is incorporated herein in its entirety by reference.

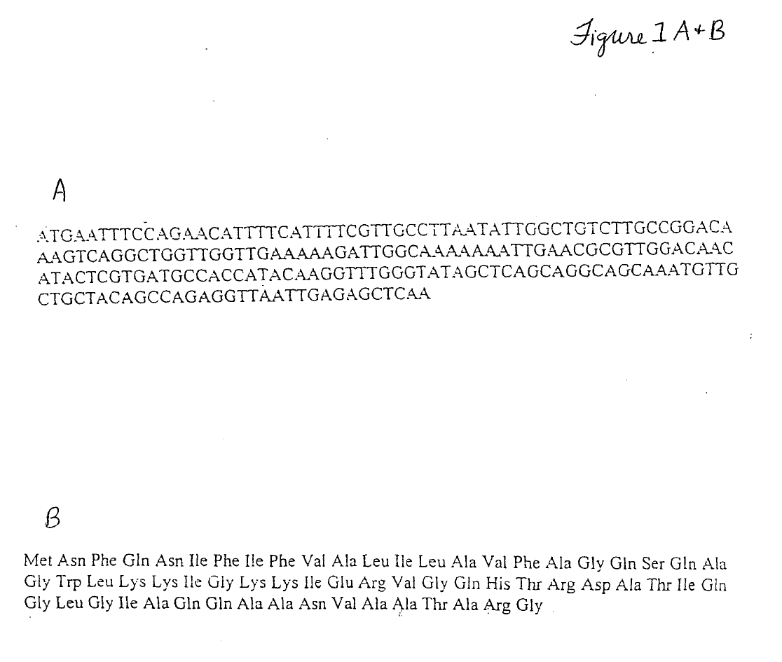

In a preferred embodiment of the invention, the lytic toxin that is so inserted and expressed is sarcotoxin IA, the gene (SEQ ID NO:1) and polypeptide (SEQ ID NO:2) sequences of which are given in FIG. 1A and B, respectively. However, those of skill in the art will recognize that many modifications of the depicted sequences may be made that would still result in a gene/polypeptide that would be suitable for use in the present invention. For example, alterations in the DNA sequence may be made for any of several reasons (for example, to produce a convenient restriction enzyme site) without affecting the amino acid sequence of the translation product. Alternatively, changes may be made which alter the amino acid sequence of the polypeptide (either purposefully to change the polyepeptide sequence, or inadvertently due to a desired change in the DNA sequence) which still result in the production of a suitable, functional polypeptide. For example, conservative amino acid substitutions may be made, or less conservative changes such as the deletion or insertion of amino acids, may be carried out. For example, amino acids may be deleted from the amino or carboxy terminus of the polypeptide, or new sequences (e.g. targeting sequences) may be added to the polypeptide. All such changes are intended to be encompassed by the present invention, so long as the resulting polypeptide is functionally expressed in the transgenic host plant and confers resistance to parasitic plants on the transgenic host plant. In general, such changes will result in a polypeptide with about 85 to 100% homology to the naturally occurring polypeptide, and preferably with about 95% homology. The amino acid homology of peptides can be readily determined by contrasting the amino acid sequences thereof as is known in the art.

In a preferred embodiment of the present invention, the gene includes a targeting sequence which directs the protein to be secreted from the cell.

The methodology for creating transgenic plants is well developed and well known to those of skill in the art. For example, dicotyledon plants such as soybean, squash, tobacco (Lin et al. 1995), and tomatoes can be transformed by Agrobacterium-mediated bacterial conjugation. (Miesfeld, 1999, and references therein). In this method, special laboratory strains of the soil bacterium Agrobacterium are used as a means to transfer DNA material directly from a recombinant bacterial plasmid into the host cell. DNA transferred by this method is stably integrated into the genome of the recipient plant cells, and plant regeneration in the presence of a selective marker (e.g. antibiotic resistance) produces transgenic plants.

Alternatively, for monocotyledon plants, such as rice (Lin and Assad-Garcia, 1996), corn, and wheat which may not be susceptible to Agrobacterium-mediated bacterial conjugation, TIMs may be inserted by such techniques as microinjection, electroporation or chemical transformation of plant cell protoplasts (Paredes-Lopez, 1999 and references therein), or particle bombardment using biolistic devices (Miesfeld, 1999; Paredes-Lopez, 1999; and references therein). Monocotyledon crop plants have now been increasingly transformed with Agrobacterium (Hiei, 1997) as well.

In order to insert a gene encoding a cecropin polypeptide into a host plant, the gene may be incorporated into a suitable construct such as a vector. Techniques for manipulating DNA sequences (e.g. restriction digests, ligation reactions, and the like) are well known and readily available to those of skill in the art. For example, Sambrook et al. 1989. Suitable vectors for use in the methods of the present invention are well known to those of skill in the art.

Further, such vector constructs may include various elements that are necessary or useful for the expression of the gene. Examples of such elements include promoters, enhancer elements, terminators, targeting sequences, and the like. Any such useful element may be incorporated into the constructs which house the lytic toxin genes used in the practice of the present invention.

In a preferred embodiment of the instant invention, the promoter which is used to direct the expression of the lytic toxin within the transgenic host plant is an inducible promoter capable of regulating intense, localized expression of the lytic toxin in the area of invasion of the parasitic plant. The promoter is operably linked to the gene. In a preferred embodiment, the promoter is located upstream of the expressible lytic toxin gene, and most preferably upstream and within about one hundred base pairs of the gene. If the gene construct includes additional elements such as targeting sequences, the promoter may be located preferably within about one hundred base pairs of such sequences. The promoter may, for example, be induced by the presence of the parasite itself, or may be selectively induced in a certain area of the plant. Examples of promoter gene regulatory sequences that are effective in directing correct expression of the lytic peptide for conferring parasite resistance on crop plants include but are not limited to:

The HMG2 promoter: HMG2 was identified in studies of the molecular basis of host-pathogen interactions in tomato (Park et al. 1992). This gene is one of four differentially-regulated genes in tomato that encode 3-hydroxy-3-methylglutaryl CoA reductase (HMGR), considered the rate limiting enzyme in the isoprenoid biosynthetic pathway (Chappell 1995). HMG2 is specifically activated during defense responses associated with the production of sesquiterpene phytoalexins (Cramer et al. 1993; Chappell et al. 1995). It has been demonstrated that parasitization by Orobanche induces expression of HMG2, in transgenic tobacco (Westwood et al. 1998). Expression of the HMG2 gene in tobacco was detected within 1 day following penetration of O. aegyptiaca, and was localized to the region around the site of the parasite invasion. Expression intensified during early Orobanche development and continued over the course of four weeks, so it does not represent a transient response to host injury. Each point of parasitic attachment induced expression independent of neighboring attachments, indicating that the HMG2 response was neither induced nor repressed by expression in nearby tissues. Indeed, incidents of secondary parasitization, where secondary roots of the parasite contacted the host root at a distance from the primary attachment site, stimulated new, localized expression. The HMG2 expression pattern in response to Orobanche represents many desirable traits of an optimal promoter for engineering resistance: expression is induced early in response to penetration of the host root, occurs in the area immediately surrounding the point of attachment, and continues throughout development of the parasite.

The HMG2 promoter, which is a preferred promoter for the practice of the present invention, is described in detail in U.S. Pat. No. 5,689,056, the complete contents of which is herein incorporated by reference.

The FTb promoter: It has been demonstrated that demonstrated that a pea (Pisum sativum L.) protein-farnesyltransferase (FTb) β-subunit promoter:GUS fusion is induced in transgenic tobacco in response to parasitization by Orobanche. Plant protein-farnesyltransferase, which post-translationally modifies signaling proteins, is important in cell cycle control and in nutrient partitioning (Qian et al., 1996; Zhou et al., 1997). Parasite induction of this promoter is consistent with Orobanche acting as a strong sink on the host root (Aber et al. 1983; Press 1995) and represents an expression pattern distinct from hmg2. FTb:GUS expression is not wound-inducible or defense-related. Rather, FTb:GUS is expressed at points of vascular intersection such as petiole branch points, the root-shoot transition zone, and secondary root junctions, consistent with a role in nutrient allocation (Zhou et al., 1997). More importantly, expression is associated with vascular tissue, appearing to be concentrated around phloem. This pattern of expression is preserved in response to parasitism by Orobanche, where expression is not observed as early as that of hmg2, but appears after the formation of vascular connections and is expressed asymmetrically around the point of attachment. The expression is initially concentrated in the stele, and above the point of parasite junction in a pattern strikingly similar to that observed at secondary root branches. As with hmg2, the FTb gene is strongly expressed throughout the development of the parasite.

Those of skill in the art will recognize that a plethora of parasitic plants exist for which there is a need to develop resistance in plants. Examples of such parasitic plants include but are not limited to facultative parasites such as Triphysaria species (for example T. versicolor); obligate hemiparasites such as Striga species (e.g. S. asiatica, S. hermonthica) and Alectra species (A. vogelii, A. picta), mistletoes such as Arceuthobium species (for example, A. americanum, A. douglasii) Phoradendron (for example, P. serotinum, P. pauciflorum); and Viscum (for example V. album, V cruciatum); and obligate holoparasites such as Orobanche (e.g. O. aegyptiaca, O. ramosa, O.crenata, O. cumana, O. cernua, O. minor) and some Cuscuta species(e.g. C. campestric, C. reflexa).

Likewise, there exist many host plants which could benefit by being transformed by the methods of the present invention to exhibit resistance to parasitic plants. Such plants include both mono- and dicotyledon species. While the practice of the present invention is applicable to all plant species, it is especially useful for crop plants such as tomato, potato, tobacco, broadbean, pepper, sunflower, parsley, carrot, lentil, eggplant, and the like.

### EXAMPLES

### Example 1

### Effect of Direct Application of SLP to Parasitic Plant Seeds

The effect of the direct application of the lytic toxin Sarcotoxin IA (SLP) to seeds of the parasitic plant O. aegyptiaca was assayed. SLP was obtained by production in S. cerevisiae, and applied to seeds during both preconditioning and germination stages as follows: Yeast strain Y426-MATa yeast cells transformed with a yeast shuttle vector-pFL61 to express the sarcotoxin IA were cultured in -URA liquid media at 30° C. for 72 h as described in Aly et al. (1999). The cultures were then centrifuged for 5 min at 4000 g and the supernatant collected for SLP assay. Quantitative evaluation of the SLP concentration present in the growth media was obtained by Western blot analysis. The effect of SLP on O. aegyptiaca seeds was determined in a Petri dish assay. Parasite seeds were surface-sterilized, dispersed on Whatman GF/A glass-fiber (0.7 cm diam.), covered with the same filter, and placed in a Petri dish. Various concentrations of SLP (0-1.4 μg/ml) or control media containing the synthetic germination stimulant1 mg/L GR24 (Mangnus et al. 1992) were added to the glass-fiber disks. After incubation in dark at 26° C. for 7 days, seeds were rated for radicles damage. The presence of SLP resulted in significantly reduced radicle elongation and seed germination of the parasite, as is shown in Table 1.

TABLE 1
Effect of sarcotoxin IA (SLP) applied at preconditioning
and at germination, on seed germination and radicle
elongation of the parasitic plant Orobanche aegyptiaca.

| | SLP Concentration | Gemination | Radicle length |
|---|---|---|---|
| Stage | (μM) | (% of control) | (% of control) |
| Preconditioning¹ | 0 | 97 ± 2^{a} | 93 ± 4 |
| | 10 | 76 ± 3 | 65 ± 6 |
| | 20 | 69 ± 6 | 52 ± 2 |
| | 30 | 6 ± 1 | 12 ± 8 |
| | 40 | 0 | 0 |
| Germination² | 0 | 96 ± 3 | 94 ± 4 |
| | 10 | 80 ± 6 | 65 ± 5 |
| | 20 | 76 ± 8 | 47 ± 3 |
| | 30 | 43 ± 7 | 33 ± 4 |
| | 40 | 36 ± 2 | 4 ± 1 |

¹SLP was mixed with the synthetic strigol (Cook et al. 1972) analogue (GR24) then applied to each disk.
²SLP was added to each disk three days after GR24 application, as soon as the seeds began to germinate.
^{a}Values are the means of two separate experiments ± the Standard error of 8 replicates.

This example demonstrates that the lytic toxin Sarcotoxin IA (SLP) inhibited seed germination and radicle elongation of the parasitic plant O. aegyptiaca.

### Example 2

### Ability of Host-Synthesized SLP to Confer Enhanced Resistance to Plant Parasites

The ability of host-synthesized SLP to confer enhanced resistance to O. aegyptiaca in potato cultivars was assayed. Chimeric genes that placed the sarcotoxin IA gene under control of the root-specific Tob promoter (Mahler-Slasky et al. 1996) were constructed as follows: The sarcotoxin IA gene fragment (327 bp) from Sarcophaga peregrina (Aly et al. 1999), cloned into PET3 plasmid as a NdeI—SstI fragment, was used as the starting point for all future constructs. Using this template the gene was amplified by PCR with the following oligonucleotides:

| | | |
|---|---|---|
| Sarco1: | 5′-GCAGGTACCATATGAATTTCCAGAAC-3′, | (SEQ ID NO:3) |
| and | | |
| Sarco2: | 5′-CTAGAGCTCT CAACCTCC TCTGGCTGTAGCAGC-3′. | (SEQ ID NO:4) |

These primers generate flanking restriction sites for the restriction enzymes KpnI (5′underlined) and SstI (3′ underlined) in the sarcotoxin IA gene to facilitate subcloning. The resulting PCR product (209 bp), which corresponds to the mature peptide and the signal peptide, was digested with KpnI and SstI, and gel purified. A plasmid containing the Tob promoter with an omega (Ω) translational enhancing sequence was digested with HindIII and KpnI, and a tri-ligation reaction performed to subclone the two genes into the pBC plasmid cut with HindIII and SstI. The identity and junctions of this construct was confirmed by sequencing. In preparation for plant transformation, the gene constructed was subcloned into an Agrobacterium tumefaciens vector pBIB_{hyg} (Becker, 1990). This vector contains the appropriate border sequence to aid in the transfer of T-DNA into plant genome and antibiotic hygromycin gene to allow selection of transgenic plants on selective medium. Potato leaves containing 1 cm of petiole were peeled with a blade containing one colony of A. tumefaciens strain LBA4404 harboring the gene construct. Potato leaves were placed on Murashige-Skoog (MS) medium for 3-4 days at 24° C., 24h light. Explants were then transferred to regeneration medium (MS salts, Benzylaminopurine 1.0 mg/L, Naphthalene acetic acid 0.1 mg/L) containing 100 mg/L hygromycin for selection of transformants and 500 mg/L carbenicillin to kill the Agrobacterium. Individual shoots were excised and transferred to rooting medium (Identical to germination medium). Rooted plantlets were then transferred to soil.

Potato cv “Desiree” was transformed with this construct and root extracts from these plants showed the presence of sarcotoxin IA by Western blot when reacted with polyclonal anti-sarcotoxin antibodies. Transgenic potato plants expressing the sarcotoxin IA gene were grown either in polyethylene bags (Hershenhorn et al. 1998) containing O. aegyptiaca seeds or in 10 liter pots containing soil artificially infested with O. aegyptiaca seeds (40 mg seeds/kg soil). Results were evaluated 60 days after growing in a greenhouse. The results from pot experiments indicate that sarcotoxin-expressing potato plants had reduced levels of Orobanche parasitism compared to nontransformed control plants. In the polyethylene bag system, where tubercle development can be visualized using a stereomicroscope, it was observed that most tubercles attached to the transgenic potato roots turned necrotic and development was abnormal.

In contrast, the SLP-expressing potatoes showed normal growth and development, suggesting the toxin is not deleterious to the host. Although the level of sarcotoxin in the roots of these transgenic potatoes was low, these results indicate that SLP produced in plant cells contacts an attached Orobanche tubercle and possesses specific anti-parasitic plant activity.

This example demonstrates that constitutive expression of the lytic toxin sarcotoxin IA gene in roots of transgenic potato plants reduces parasitism by O. aegyptiaca.

### Example 3

### Characterization of the Response of Transgenic Tomato and Tobacco to Orobanche in the Lab and Greenhouse.

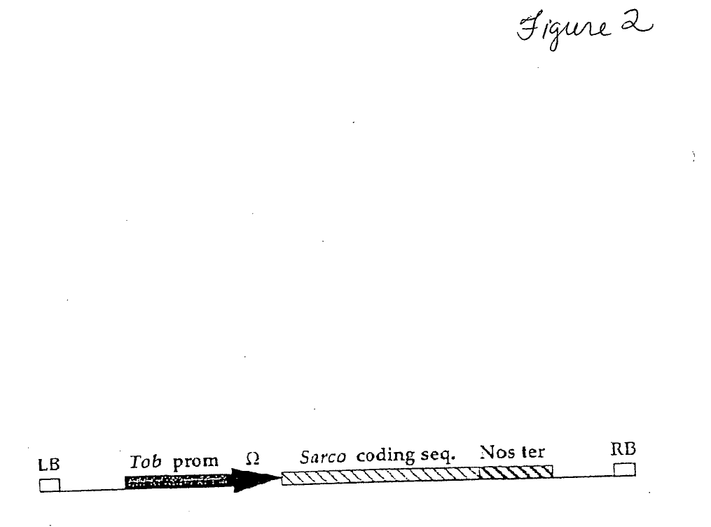

The construct depicted in FIG. 2 was used to produce tobacco and tomato transgenic plants by methods identical to those described above. The construct contained the root-specific promoter (Tob), the translational enhancing sequence (Ω), sarcotoxin coding sequences and the nopaline synthase (Nos) terminator. The construct was cloned into a pGA492 binary vector and transformed to Agrobacterium for plant transformation. Results in Tobacco:

Following transformation and selection of tobacco (Xanthi) discs, 15 putative transgenic tobacco plants (T₁) were selected and transferred to small pots, then to 10-liter pots containing soil highly inoculated with O. aegyptiaca. Controls consisted of non-transgenic plants in soil either inoculated or not inoculated with O. aegyptiaca. Although few of the fist generation of transgenic tobacco plants survived, the seeds produced by these plants were used in subsequent analysis. For the next generation (T₂), 70 putative transgenic plants were grown and transplanted into 10-liter pots with infested soil to select the O. aegyptiaca resistant plants. In parallel, the presence of the sarcotoxin transgene was determined in the leaves of transgenic tobacco using Southern blot analysis.

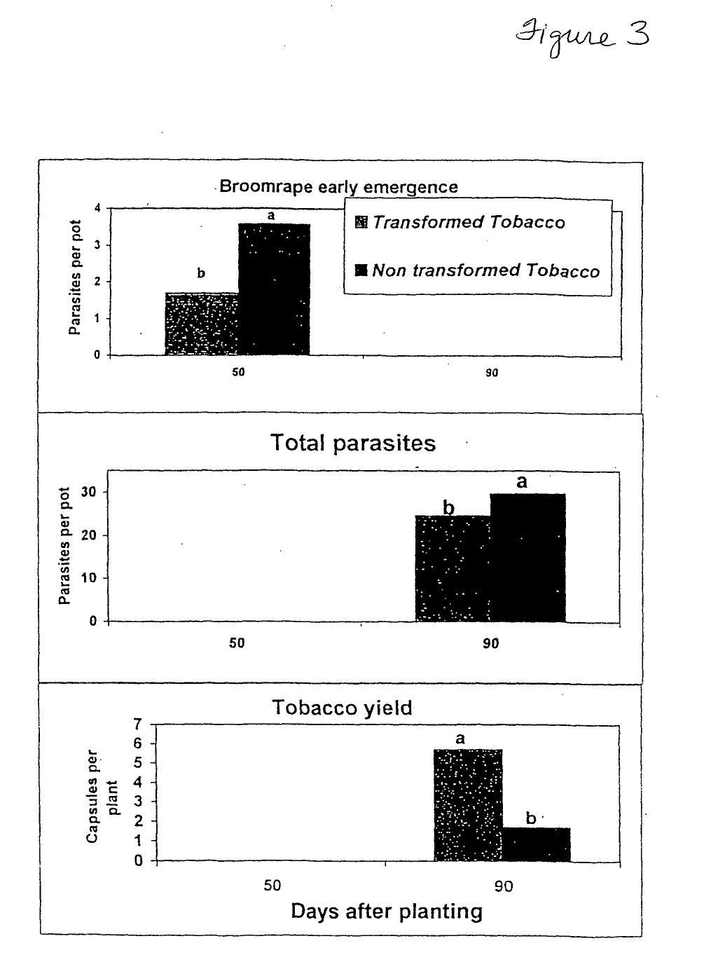

Results from testing the T₂ generation indicated that the transgenic tobacco plants expressing sarcotoxin IA gene showed significantly reduced O. aegyptiaca growth and increased tobacco yield as compared to non-transformed control plants (FIG. 3). Some transgenic plants showed exceptionally high resistance, and O. aegyptiaca on these plants were unable to develop normally as evidenced by the inflorescence shoots remaining small and unhealthy compared to those parasitizing nontransgenic tobacco plants (FIG. 3). Results in Tomato:

Tomato VF-6 disc plants were transformed with Agrobacterium harboring the sarcotoxin gene (using the same construct as with the tobacco, depicted in FIG. 2). From 15 putative transgenic (T₁) plants, only two were able to produce fruits. Seeds were collected from these and replanted into 10-liter pots without O. aegyptiaca in order to multiply seed for further analysis. Analysis of these plants revealed lines with varying level of resistance to Orobanche, but all were significantly more resistant than non-transformed control plants.

Transgenic tobacco expressing sarcotoxin IA gene reduced significantly O. aegyptiaca infestation and affected yield production in pots as compared to non-transgenic control. Transformed tomato plants (VF-6) with sarcotoxin IA gene, showed partial to absolute resistance to O. aegyptiaca parasitization in pots.

This example demonstrates that the protective effect of sarcotoxin IA against parasitism by Orobanche is applicable to different plant species and reproducible across multiple transformation events.

### Example 4

### Results from Transgenic Plants Carrying Constructs with Inducible Promoters

The results from plants containing SLP under the control of the (Tob) promoter were highly encouraging. However, this promoter directs a constant, low level of gene expression in plant roots. The efficacy of SLP can be increased by fusing it to promoters that are expressed strongly and specifically in the area of parasite attachment. Thus, two gene promoters previously shown to be Orobanche-inducible were tested: HMG2 (from tomato 3-hydroxy-3-methylglutaryl CoA reductase) and FTb (from pea farnesyltransferase).

Generation of constructs consisting of SLP (0.3 kb) fused to HMG2 promoter (0.4 kb) fragment was performed using pBC cloning vector to facilitate efficient clone recovery and sequence confirmation. The sarcotoxin IA genes was amplified by PCR as described above, digested with HindIII/SstI to create flanking restriction sites. A PCT151 plasmid containing the HMG2 promoter was digested with HindIII and KpnI, and a tri-ligation reaction performed to subclone the two genes into the pBC plasmid cut with HindIII and SstI. Once the constructs were confirmed in E. coli, and were found in the right orientation by sequence analysis, they were successfully mobilized into Agrobacterium tumefaciens strain GV3101 and confirmed by reisolation and restriction analysis.

Arabidopsis (Arabidopsis thaliana cv. Columbia) plants were transformed using the vacuum infiltration method of Bechtold et al. (1993). Plants were grown to the stage at which they just started to flower and the flowers were then immersed for 15 min in a suspension of Agrobacterium strain GV3101 harboring the gene construct. Plants were maintained for 2-3 weeks until mature and seeds were collected. Progeny seeds were harvested, surface sterilized, and then germinated on a medium of MS salts (Murashige and Skoog 1962) containing the antibiotic hygromycin (40 mg/L) for selection. Putatively transformed plants generated for the HMG2:SARCOTOXIN IA gene construct were grown in individual pots in a growth chamber under a 16 h light/8 h dark regime. Progeny of these plants were subsequently selected again on hygromycin media and the T₂ generation tested for resistance to O. aegyptiaca.

Arabidopsis seeds (60 per pot) carrying the HMG2:SARCOTOXYIN IA gene construct were planted in potting mix (Metro Mix 360) inoculated with 5 mg O. aegyptiaca seed per ml volume. Plants were grown in the greenhouse along side similar pots containing wild type Arabidopsis plants growing in either inoculated or non-inoculated soil. The primary effect of Orobanche parasitism is to delay and decrease the reproductive capacity of the host plant, so visual observations were made with respect to plant vigor and time of flowering.

Table 2 shows the results of this experiment. Plant vigor was rated 34 days after planting, when difference in plant size and pigmentation were evident (Arabidopsis increases is flavonoids when under stress, taking on a puple color). All of the lines containing the SLP transgene (L15-L95) appeared significantly healthier than the inoculated nontransgenic line, and at 40 days after planting most were at least equal to the control plants. The time of flowering reflected this trend, with transgenic plants flowering simultaneously or slightly after the non-inoculated control plants, and clearly ahead of the inoculated non-transformed plants. Some of the variability in this experiment may be attributed to some percentage of nontransformed plants among the lines (they were not confirmed to be homozygous for the transgene) or variation in levels of transgene expression. Nevertheless, these results indicate clear differences in susceptibility to parasitism by O. aegyptiaca.

These results demonstrate that the sarcotoxin IA gene product is effective in increasing resistance to parasitism in yet another plant species. Given these results it is reasonable to generalize that sarcotoxin IA is effective in conferring resistance to Orobanche species in multiple host plants. They also demonstrate the efficacy of sarcotoxin LA under control of a second promoter.

TABLE 2

| Line | Vigor* | Flowering (%)** |
|---|---|---|
| Wild type non-inoculated | 10 | 50 |
| Wild type inoculated | 4 | 3 |
| L15 | 10 | 45 |
| L19 | 8 | 20 |
| L23 | 7 | 18 |
| L25 | 7 | 23 |
| L35 | 10 | 55 |
| L70 | 10 | 35 |
| L95 | 10 | 48 |

*Vigor was rated visually on a scale of 1-10, with 1 being dead and 10 being completely healthy. The wild type non-inoculated plants were designated as 10.
**Percentage of emerged plants that were flowering or had initiated a clearly visible floral shoot.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### References

Aber, M., A. Fer, and G. Salle. 1983. Z. Pflaizenphysiol. Bd. 112:297-308.

Aly, R., D. Granot, Y. Mahler-Slasky, N. Halpern, and E. Galun. 1999. Protein Expression and Purification 16, 120-124.

Boman, H. G. and D. Hultmark. 1987. Annu. Rev. Microbiol. 41:103-126.

Chappell, J. 1995. Annu. Rev. Plant Physiol. Plant Mol. Biol. 46:521-548.

Clark., D. P. et al., J. Biol. Chem., 269:10849-10855 (1994)

Cramer, C. L., D. Weissenborn, C. K. Cottingham, C. J. Denbow, J. D. Eisenback, D. N. Radin, and X. Yu. 1993. J. Nematol. 25:507-518.

Cubero, J. I. 1991. Breeding for resistance to Orobanche species: a review. pp. 257-277 In: K. Wegmann and L. J. Musselman, eds. Progress in Orobanche Research. Eberhard-Karls-Universität, Tübingen, Germany.

DePamphilis, D. W., N. D. Young, and A. D. Wolfe. 1997. Proc. Natl. Acad. Sci. USA 94:7367-7372.

Düring, K. 1996. Mol. Breed. 2:297-305.

Ejeta, G., L. G. Butler, D. E. Hess, and R. K. Vogler. 1991. Genetic and breeding strategies for Striga resistance in sorghum. pp. 539-544 In: J. K. Ransom, L. J. Musselman, A. D. Worsham, and C. Parker, eds., Proc. 5th Intl. Symp. Parasitic Weeds. CIMMYT, Nairobi, Kenya.

ENBbuDePamphilis, C. W. and J. D. Palmer. 1990. Nature 348:337-339.

English, T. J., R. S. Norris, and W. K. Jones. Weed Sci. Soc. of Amer. Abstracts 38:28.

Eplee, R. and M. Langston. 1991. Integrated control methodology for Striga in the USA. Pp. 397-399 In: J. K. Ransom, L. J. Musselman, A. D. Worsham, and C. Parker, eds., Proc. 5th Intl. Symp. Parasitic Weeds. CIMMYT, Nairobi, Kenya.

Foy, C. L., R. Jain, and R. Jacobson. 1989. Rev. Weed Sci. 4:123-152.

Frost, C. C. and L. J. Musselman. 1980. Clover broomrape (Orobanche minor) in the United States. Weed Sci. 28. Gressel, J., L. Segel, and J. K. Ransom. Int. J. Pest Manag. 42:113-129.

Hershenhorn, J., D. Plakhine, Y. Goldwasser, J. Westwood, C. L. Foy, and Y. Kleifeld. 1998. Weed Technol. 12:108-114.

Hiei, Y et al. Plant Mol. Biol. 1997,35:205-218.

Jacobsohn, R. 1994. The broomrape problem in Israel and an integrated approach to its control. pp. 652-658 In: A. H. Pieterse, J. A. C. Verkleij, and S. J. ter Borg, eds., Biology and Management of Orobanche, Proc. of the 3rd Int. Workshop on Orobanche and Related Striga Research. Royal Tropical Inst., Amsterdam, The Netherlands.

Jaynes, J. M., G. R. Julian, G. W. Jeffers, K. L. White, and F. M. Enright. 1989. Pept. Res. 2:157-160.

Joel, D. M., Y. Kleifeld, D. Losner-Goshen, G. Herzlinger, and J. Gressel. 1995. Nature 374:220-221.

Kanai, A. and S. Natori. 1989. FEBS Lett. 258:199-202.

Lin, J.-J. and Assad-Garcia, In Vitro 1996; 32:35A-36A.

Lin, J.-J., Assad-Garcia, N. and Kuo, J. Plant Science 1995; 109:171-177.

Mahler-Slasly, Y., S. Galili, A. Perl, R. Aly, S. Wolf, D. Aviv, and E. Galun. 1996. Root directed expression of alien genes in transgenic potato: sarcotoxin and Gus. pp.187-192 In: A. Altman and Y. Waisel, eds., Biology of Root Formation. Plenum Press, N.Y.

Mangnus E. M., Stommen P. L. A., Zwanenburg B. 1992. J Plant Growth Regul 11: 91-98

Miesfeld, R. L. Applied Molecular Genetics, 1999; Wiley-Liss, publisher, pp.205-235.

Minet, M., M. E. Dufour, and F. Lacroute. 1992. Plant J. 2:417-422.

Morikawa, N. et al., Biochem. Biophys. Res. Commun., 189:184-190(1992)

Natori, S. 1995. Nippon Rinsho 53:1297-1304.

Nordeen, R. D., S. L. Sinden, J. M. Jaynes, and L. D. Owens. 1992. Plant Sci. 82:101-107.

Okada, M. and S. Natori. 1985. Biochem. J. 229:453-458.

Paredes-Lopez, ed. Molecular Biotechnology for Plant Food Production, Technomic Publishing, Inc. 1999; 83-86.

Park, H., C. J. Denbow, and C. L. Cramer. 1992. Plant Mol. Biol. 20:327-331.

Parker, C. and A. K. Wilson. 1986. FAO Plant Prot. Bull. 34:83-98.

Parker, C. and C. R. Riches. 1993. CAB Int., Wallingford, UK.

Press, M. C. 1995. Carbon and nitrogen relations. pp. 103-124. In: M. C. Press and J. D. Graves, eds., Parasitic Plants. Chapman and Hall, London.

Qian, D., D. Zhou, R. Ju, C. L. Cramer, and Z. Yang. 1996. Plant Cell 8:2381-2394.

Sauerborn, J. 1991. The economic importance of the phytoparasites Orobanche and Striga. pp. 137-143 In: J. K. Ransom, L. J. Musselman, A. D. Worsham, and C. Parker, eds., Proc. 5th Intl. Symp. Parasitic Weeds. CIMMYT, Nairobi, Kenya.

Simmaco, M. et al., FEBS Lett., 324:159-161(1993)Surov, T., D. Aviv, R. Aly, D. M. Joel, T. Goldman-Gues, and J. Gressel. 1998. Theor. Appl. Genet. 96:132-137.

Westwood, J. H., X. Yu, C. L. Foy, and C. L. Cramer. 1998b. MPMI 11:530-536. Zhou, D., D. Qian, C. L. Cramer, and Z. Yang. 1997. Plant J. 12:921-930.

Zasloff, M., Proc. Natl. Acad. Sci., USA, 84:5449-5453(1987)

We claim:

1. A transgenic plant protected from parasitic plants, comprised of a host plant harboring an expressible gene encoding a lytic toxin that inhibits attack from parasitic plants.

2. The transgenic plant of claim 1 wherein said host plant is a dicotyledon.

3. The transgenic plant of claim 2 wherein said host plant is a tomato.

4. The transgenic plant of claim 2 wherein said host plant is a potato.

5. The transgenic plant of claim 2 wherein said host plant is tobacco.

6. The transgenic plant of claim 1 wherein said expressible gene encodes a cecropin.

7. The transgenic plant of claim 6 wherein said expressable gene is SEQ ID NO:1.

8. The transgenic plant of claim 1 wherein said parasitic plant is selected from the group consisting Triphysaria, Striga, Alectra, Arceuthobium, Phoradendron, Viscum, Orobanche and Cuscuta.

9. The transgenic plant of claim 8 wherein said parasitic plant is of the genus Orobanche.

10. The transgenic plant of claim 9 wherein said parasitic plant is Orobanche aegyptiaca.

11. The transgenic plant of claim 9 wherein said parasitic plant is Orobanche minor.

12. The transgenic plant of claim 1 further comprising an inducible promoter operably linked to said expressible gene encoding a lytic toxin which promotes localized expression of said expressible gene in the area of invasion of said parasitic plant.

13. The transgenic plant of claim 12 wherein said inducible promoter is a parasite induced promoter.

14. The transgenic plant of claim 12 wherein said inducible promoter is a root-specific promoter.

15. The transgenic plant of claim 12 wherein said inducible promoter is located on a DNA molecule on which said inducible promoter is located at a location upstream of said expressible gene.

16. The transgenic plant of claim 12 wherein said location is within one hundred base pairs of said expressible gene.

17. A method for protecting plants from damage caused by parasitic plants, comprising the step of providing a host plant with an expressable gene encoding a lytic toxin which produces a polypeptide that inhibits attack from parasitic plants.

18. The method of claim 17 wherein said providing step includes providing an inducible promoter operably linked to said expressible gene which promotes localized expression of said expressible gene in the area of invasion of said parasitic plant.

19. A method of preventing or reducing damage in a host plant which may be attacked by a parasitic plant, comprising the step of harboring in said host plant an expressible gene encoding a lytic toxin that inhibits attack from parasitic plants.

20. The method of claim 19 wherein said host plant is a dicotyledon.

21. The method of claim 20 wherein said host plant is a tomato.

22. The method of claim 20 wherein said host plant is a potato.

23. The method of claim 20 wherein said host plant is tobacco.

24. The method of claim 19 wherein said expressible gene encodes a cecropin.

25. The method of claim 20 wherein said expressible gene is SEQ ID NO:1.

26. The method of claim 19 wherein said parasitic plant is selected from the group consisting of Triphysaria, Striga, Alectra, Arceuthobium, Phoradendron, Viscum, Orobanche and Cuscuta.

27. The method of claim 26 wherein said parasitic plant is of the genus Orobanche.

28. The method of claim 27 wherein said parasitic plant is Orobanche aegyptiaca.

29. The method of claim 27 wherein said parasitic plant is Orobanche minor.

30. The method of claim 29 wherein said providing step includes providing an inducible promoter operably linked to said expressible gene which promotes localized expression of said expressible gene in the area of invasion of said parasitic plant.

31. The transgenic plant of claim 30 wherein said inducible promoter is a parasite induced promoter.

32. The transgenic plant of claim 30 wherein said inducible promoter is a root-specific promoter.

33. A potato plant transformed by an expressible gene encoding a lytic toxin which produces a polypeptide that inhibits attack from parasitic plants.

34. The potato plant of claim 33 wherein said expressible gene is SEQ ID NO: 1.

35. A tomato plant transformed by an expressible gene encoding a lytic toxin which produces a polypeptide that inhibits attack from parasitic plants.

36. The tomato plant of claim 35 wherein said expressible gene is SEQ ID NO: 1.

37. A tobacco plant transformed by an expressible gene encoding a lytic toxin which produces a polypeptide that inhibits attack from parasitic plants.

38. The tobacco plant of claim 37 wherein said expressible gene is SEQ ID NO: 1.

39. A host plant having a root system containing a polypeptide of SEQ ID NO:2.


SEQUENCE LISTING (4 sequences)

SEQ ID NO: 1 - DNA, 203 bp, Sarcophaga peregrina
atgaatttccagaacattttcattttcgttgccttaatattggctgtcttgccggacaaagtcaggctggttggttgaaaaagattggcaaaaaaattgaacgcgttggacaacatactcgtgatgccaccatacaaggtttgggtatagctcagcaggcagcaaatgttgctgctacagccagaggttaattgagagctcaa

SEQ ID NO: 2 - protein, 63 aa, Sarcophaga peregrina
MNFQNIFIFVALILAVFAGQSQAGWLKKIGKKIERVGQHTRDATIQGLGIAQQAANVAATARG

SEQ ID NO: 3 - DNA, 26 bp, Artificial sequence
  Synthetic oligonucleotide primer
gcaggtaccatatgaatttccagaac

SEQ ID NO: 4 - DNA, 33 bp, Artificial sequence
  Synthetic oligonucleotide primer
ctagagctctcaacctcctctggctgtagcagc